(19) United States
(12) Patent Application Publication
(10) Pub. No.: US 2025/0195352 A1
(43) Pub. Date: Jun. 19, 2025

(54) LIPID FORMULATIONS CONTAINING BIOACTIVE FATTY ACIDS AND OTHER BIOACTIVE AGENTS

(71) Applicant: Sciadonics, Inc., Long Lake, MN (US)

(72) Inventor: Alvin Berger, Long Lake, MN (US)

(21) Appl. No.: 18/845,514

(22) PCT Filed: Mar. 10, 2023
(86) PCT No.: PCT/US2023/014947

§ 371 Date: Sep. 10, 2024

Related U.S. Application Data
(60) Provisional application No. 63/318,589, filed on Mar. 10, 2022.

(51) Int. Cl. A61K 8/36; A61K 31/202; A61P 29/00; A61Q 17/00

(52) U.S. Cl. CPC A61K 8/361; A61K 31/202; A61P 29/00; A61Q 17/00

(57) ABSTRACT

Provided herein is technology relating to compositions containing bioactive non-methylene interrupted fatty acids (NMIFAs) for use in improving skin barrier function and related diseases and disorders.

### RELATED APPLICATIONS

The present application claims priority to U.S. Provisional Application No. 63/318,589, filed Mar. 10, 2022, which is incorporated herein by reference in its entirety.

### FIELD OF TECHNOLOGY

Provided herein is technology relating to compositions containing bioactive non-methylene interrupted fatty acids (NMIFAs) for use in improving skin barrier function and related diseases and disorders.

### BACKGROUND OF THE INVENTION

Sciadonic acid (SA) (all cis Δ5,11,14 eicosatrienoate) is principally found in various gymnosperm plant species. Its structure and example of an eicosanoid metabolite are shown in FIG. 1. SA is an anti-inflammatory fatty acid analog of arachidonic acid (5,8,11,14 eicosatetraenoate; ARA) lacking the Δ-8 double bond necessary for formation of typical pro-inflammatory, fully-formed, eicosanoid mediators. Our group and others have shown SA to exert anti-inflammatory actions by reducing levels of ARA and downstream mediators in in vitro and animal models. See, e.g., Berger A, Monnard I, Baur M, Charbonnet C, Safonova I, Jomard A. Epidermal anti-Inflammatory properties of 5, 11, 14 20:3: effects on mouse ear edema, PGE2 levels in cultured keratinocytes, and PPAR activation. Lipids Health Dis. 2002; 1:5; Butani L, Afshinnik A, Johnson J, Javaheri D, Peck S, German J B, et al. Amelioration of tacrolimus-induced nephrotoxicity in rats using juniper oil. Transplantation. 2003; 76(2):306-11; Chen S J, Huang W C, Yang T T, Lu J H, Chuang L T. Incorporation of sciadonic acid into cellular phospholipids reduces pro-inflammatory mediators in murine macrophages through NF-kB and MAPK signaling pathways. Food Chem Toxicol. 2012; 50(10):3687-95; Ells R, Kock J L, Albertyn J, Hugo A, Pohl C H. Sciadonic acid modulates prostaglandin E2 production by epithelial cells during infection with C. albicans and C. dubliniensis. Prostaglandins Other Lipid Mediat. 2012; 97(1-2):66-71; Chen S J, Chuang L T, Liao J S, Huang W C, Lin H H. Phospholipid incorporation of non-methylene-interrupted fatty acids (NMIFA) in murine microglial BV-2 cells reduces pro-inflammatory mediator production. Inflammation. 2015; 38(6):2133-45; Zhou X, Shang J, Qin M, Wang J, Jiang B, Yang H, et al. Fractionated antioxidant and anti-inflammatory kernel oil from Torreya fargesii. Molecules. 2019; 24(18):3402; Pedrono F, Boulier-Monthean N, Boissel F, Ossemond J, Viel R, Fautrel A, et al. Sciadonic acid derived from pine nuts as a food component to reduce plasma triglycerides by inhibiting the rat hepatic Delta 9-desaturase. Sci Rep. 2020; 10(1):6223; Baker E J, Miles E A, Calder P C. A review of the functional effects of pine nut oil, pinolenic acid and its derivative eicosatrienoic acid and their potential health benefits. Prog Lipid Res. 2021; 82:101097.

Topical anti-inflammatory properties using lipidic seed extracts rich in SA have also been demonstrated [1, 6, 9-11]. See, e.g., Berger et al. 2002 and Zhou et al. 2019, supra; Mascolo N, Autore G, Capasso F, Menghini A, Fasulo M P. Biological screening of Italian medicinal plants for anti-inflammatory activity. Phytother Res. 1987; 1(1):28-31; Chen B Q, Cui X Y, Zhao X, Zhang Y H, Piao H S, Kim J H, et al. Antioxidative and acute antiinflammatory effects of Torreya grandis. Fitoterapia. 2006; 77(4):262-67; Saeed M K, Deng Y, Dai R, Li W, Yu Y, Iqbal Z. Appraisal of antinociceptive and anti-inflammatory potential of extract and fractions from the leaves of Torreya grandis Fort Ex. Lindl. J Ethnopharmacol. 2010; 127(2):414-8.

In addition to the anti-inflammatory benefits and other physiological benefits of SA (see references in previous paragraph), SA is relatively oxidatively stable. When there are two or more double bonds present in a methylene interrupted arrangement in a fatty acid molecule, in response to oxidative stress, the fatty acyl molecules are subject to hydrogen abstraction on the carbon flanked by two double bonds, and subsequent peroxidation (autoxidation). In the case of SA, only two double bonds (A11-12 and A14-15) are methylene-interrupted and subject to peroxidation. This could contribute to greater stability of SA relative to a typical triene, in which all three double bonds are methylene-interrupted (e.g., alpha linolenic acid present in linseed oil). SA can thus be added to skin formulations and food products (if consumed orally) in commercial products requiring longer shelf-lives; and with less complicated and less expensive antioxidant systems mandated.

We previously demonstrated that topically-applied methyl esters from an oil similar to Delta-5® led to SA incorporation into mouse ear phospholipids, including the important signaling phosphatidylinositol pool; and reduced phospholipid levels of ARA; and -ARA- and TPA (12-O-tetradecanoylphorbol-13-acetate)-induced mouse ear edema. See Berger et al. 2002, supra. Purified SA taken up by cultured human skin keratinocytes reduced ARA, and -pro-inflammatory prostaglandin E2 (PGE2) levels dramatically.

### SUMMARY OF THE INVENTION

Provided herein is technology relating to compositions containing bioactive non-methylene interrupted fatty acids (NMIFAs) for use in improving skin barrier function and related diseases and disorders.

In some preferred embodiments, the present invention provides NMIFA compositions for use in treating a disease or condition associated with loss of barrier function in a human or animal subject or to increase collagen production. In some preferred embodiments, the subject is need or of the treatment.

In some preferred embodiments, the disease or condition associated with loss of barrier function in a human or animal subject is selected from the group consisting of inflammation or irritation caused by an allergen, inflammation or irritation caused by a chemical irritant, inflammation or irritation caused by nickel, latex, skin disinfectants, topical corticosteroids/glucocorticoids, topical germicidal compositions, inflammation or irritation caused by plant toxins or mechanical plant irritants, inflammation or irritation caused by insect toxins, inflammation or irritation caused by exposure to an oil-in-water (O/W) emulsion, inflammation or irritation due sunburn or damage caused by UV-B exposure, inflammation or irritation due to exposure to a skin whitening, bleaching, or lightening agent, inflammation or irritation caused by environmental pollutant, dry skin due to loss of skin moisture, loss of skin elasticity, skin wrinkling, and frown lines inflammation or irritation by an allergen, inflammation or irritation by a chemical irritant, and inflammation or irritation by plant toxins, inflammation or irritation by insect toxins, dry skin due to loss of skin moisture, loss of skin elasticity, skin wrinkling, and frown lines.

In some preferred embodiments, the allergen is pet dander.

In some preferred embodiments, the chemical irritant is selected from the group consisting of bezalkonium chloride (BAC), sodium lauryl sulfate (SLS), benzoyl peroxide, a PABA-based chemical, an adhesive, a chemical fragrance, a chemical found in household cleaners (e.g., dish detergents, laundry detergent, window cleaners, furniture polish, drain cleaners and toilet disinfectants), residual solvent (e.g., xylene, toluene, and trimethylbenzene, octanoic acid, nonanoic acid, decanoic acid, and n-propanol and other alcohols) and a soap.

In some preferred embodiments, the mechanical plant irritant is selected from the group consisting of a plant thorn, plant spine, plant nettle, plant glochid, plant trichome, and sharp-edged leaves.

In some preferred embodiments, the plant toxin is selected from the group consisting of urushiol, calcium oxalate, protoanemonin, isothiocyanates, bromelain, diterpene esters, alkaloids, naphthoquinone and plant-derived acids.

In some preferred embodiments, the insect toxin is selected from the group consisting of formic acid and mosquito saliva.

In some preferred embodiments, the O/W emulsion is selected from the group consisting of Aqueous Cream BP 2001, Clioquinol Cream BP 1999 without clioquinol, Nonionic Hydrophilic Cream DAB 2001 without glycerol, Hydrophilic Skin Emulsion Base NRF, and Base Cream DAC.

In some preferred embodiments, the skin whitening, bleaching, or lightening agent is selected from the group consisting of magnesium-1-ascorbyl-2-phosphate (MAP), hydroxyanisole, N-acetyl-4-S-cysteaminylphenol, arbutin (hydroquinone-beta-d-glucopyranoside) and hydroquinone.

In some preferred embodiments, the environmental pollutant is selected from the group consisting of solar ultraviolet radiation (UVR), a polycyclic aromatic hydrocarbon (PAH), a volatile organic compounds (VOC), nitrogen oxides (NOx), particulate matter (PM), and cigarette smoke.

In some preferred embodiments, the skin surface dysbiosis-induced irritation is caused by a skin pathogen or disruption of the skin microbiome.

In some preferred embodiments, the NMIFA composition comprises at least 2% w/w, at least 5% w/w, at least 10% w/w, or at least 20% w/w of NMIFAs. In some preferred embodiments, the NMIFA composition comprises from 1% to 40% w/w, 1% to 30% w/w, 5% to 40% w/w, 5% to 30% w/w, or 10% to 25% w/w of NMIFAs. In some preferred embodiments, the NMIFAs in the NMIFA composition are selected from the group consisting of sciadonic acid, juniperonic acid, pinoleic acid, dihomopinoleic acid, coniferonic acid (5, 9, 12, 15 18:4) and synthetic fatty acids selected from the group consisting of 1, 11, 14, 17 20:4; 2, 11, 14, 17 20:4; 3, 11, 14 17 20:4; 4, 11, 14 17 20:4; 6, 11, 14 17 20:4; 7, 11, 14 17 20:4; 1, 9, 12, 15 18:4; 2, 9, 12, 15 18:4; 3, 9, 12, 15 18:4; 4, 9, 12, 15 18:4, 5, 9, 12, 15 18:4, 1, 11, 14 20:3; 2, 11, 14 20:3; 3, 11, 14 20:3; 4, 11, 14 20:3; 6, 11, 14 20:3; 1, 9, 12 18:3; 2, 9, 12 18:3; 3, 9, 12 18:3; and 4, 9, 12 18:3 fatty acids and combinations thereof. In some preferred embodiments, NMIFA is sciadonic acid. In some preferred embodiments, the NMIFAs in the NMIFA composition are provided as free fatty acids, esters, ethyl esters, triglycerides or phospholipids and combinations thereof. In some preferred embodiments, the NMIFAs in the NMIFA composition are formulated with an antioxidant not naturally occurring with the NMIFAs. In some preferred embodiments, the NMIFA composition is formulated for topical delivery. In some preferred embodiments, the NMIFA composition is formulated for oral delivery.

### BRIEF DESCRIPTION OF THE DRAWINGS

FIG. 1: Structure of sciadonic acid (SA). SA is a non-methylene interrupted fatty acid present in Delta-5® oil as a triacylglycerol, representing 20-25% of total fatty acids in the oil. It is a 20-carbon fatty acid, with 3 double bonds present in Δ-5, 11, and 14 positions in cis-configuration. As a result of having only 2 methylene-interrupted fatty acids, it has good oxidative stability like a dienoic fatty acid. In skin, sciadonic acid exerts anti-inflammatory properties by displacing arachidonic acid from lipid pools; but may also potentially form bioactive, aborted cyclooxygenase and lipoxygenase and other eicosanoid products (a very poorly studied area). In our example herein, the C13-hydrogen at the center of the 1,4 cis, cis pentadiene system is extracted, following oxygen attack at C-15, forming 15-hydroxy-5,11,13-eicosatrienoic acid by the cyclooxygenase system. Another cyclooxygenase product (not shown) is 11-hydroxy-5,12,14-eiosatrienoic acid. See Evans R W, Sprecher H. Metabolism of icosa-5,11,14-trienoic acid in human platelets and the inhibition of arachidonic acid metabolism in human platelets by icosa-5,8,14-triynoic and icosa-5,11,14-triynoic acids. Prostaglandins. 1985; 29(3):431-41. If SA were acted on by a skin 12-lipoxygenase, then 12-hydroxy-5,10,14-eicosatrienoic acid would be theoretically formed (not shown).

FIG. 2: Simplified Experimental Design. Shown are measurements and treatments applied to each subject's forearm on days 0-28, in the order conducted (top of each box, down). Test site 1, Untreated control; Site 2, Untreated control, chemically damaged with 2% SLS; and Test Site 3, Delta-5® oil applied to SLS-damaged skin. TEWL (g/m²h) and NOVA Impedance (units are in arbitrary Dermal Phase Meter impedance units; DPMIU) were measured just before SLS was applied to the skin on Day 0 (baseline); and redness (measured by Visual Expert Grading Scores; VEGS) was measuredjust after SLS was applied to the skin on Day 0*. On Day 1, redness, TEWL and impedance values are measured 1 day after SLS injury, then Delta-5® oil is applied to the Delta-5® oil group, so that on Day 2, Delta-5® oil has been on the skin for 24 h. Redness was not measured on Day 2. D5 2X/d, Delta-5® oil applied twice per day (see text for further details).

FIGS. 3a and 3b: TEWL (g/m²h) evaluated as differences from Day 1 in Delta-5® oil-SLS damaged and untreated damaged groups at various time points, with—(FIG. 3a) and without (FIG. 3b) control correction. Values represent the means of differences from each time point from day 1. Values from each time point were divided by the corresponding untreated control value in FIG. 3a. *p<0.05; **p<0.01. Percents displayed represent the percent differences between Delta-5® oil-SLS damaged and untreated damaged groups. Standard deviations are not shown for readability of the graphs; and can be found in Table 1.

FIG. 4: Impedance (Units are in arbitrary Dermal Phase Meter impedance units; DPMIU) evaluated as differences from Day 1 in Delta-5® oil- and untreated groups, shown as means at various time points. See FIG. 3 for statistical details and explanations. Standard deviations are not shown for readability of the graphs; and can be found in Table 1.

FIG. 5: Redness (Visual Expert Grading Scores; VEGS) evaluated in Delta-5® oil and untreated groups at various time points, shown as mean VEGS at each time point. Note that redness was not evaluated as differences from Day 1 for reasons explained in the text. Percents displayed represent percent differences for the means between Delta-5® oil-SLS damaged and untreated damaged groups at each time point. Standard deviations are not shown for readability of the graph but are found in the section “Results SLS study-Skin redness”. *p<0.05.

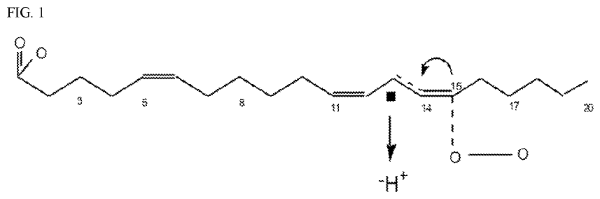

### DETAILED DESCRIPTION OF THE INVENTION

Provided herein is technology relating to compositions containing bioactive non-methylene interrupted fatty acids (NMIFAs) for use in improving skin barrier function and related diseases and disorders.

This technology is described below, wherein the section headings are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

Delta-5® oil (Delta-5) refers to the oil sold by SciaEssentials®, LLC with 20-25% SA.

As used herein, “active” or “activity” refers to native or naturally occurring biological and/or immunological activity.

As used herein the term, “in vitro” refers to an artificial environment and to processes or reactions that occur within an artificial environment. In vitro environments may include, but are not limited to, test tubes and cell cultures. The term “in vivo” refers to the natural environment (e.g., an animal or a cell) and to processes or reactions that occur within a natural environment.

As used herein, the terms “subject” and “patient” refer to any animal, such as a mammal like a dog, cat, bird, livestock, and preferably a human (e.g., a human with a disease such as obesity, diabetes, or insulin resistance).

As used herein, the term “individual” refers to vertebrates, particularly members of the mammalian species. The term includes but is not limited to domestic animals, sports animals, primates, and humans.

As used herein, the term “effective amount” refers to the amount of a composition sufficient to effect beneficial or desired results. An effective amount can be administered in one or more administrations, applications, or dosages and is not intended to be limited to a particular formulation or administration route.

As used herein, the term “administration” refers to the act of giving a drug, prodrug, or other agent, or therapeutic treatment to a subject. Exemplary routes of administration to the human body can be through the eyes (ophthalmic), mouth (oral), skin (transdermal, topical), nose (nasal), lungs (inhalant), oral mucosa (buccal), ear, by injection (e.g., intravenously, subcutaneously, intratumorally, intraperitoneally, etc.), and the like.

As used herein, the term “co-administration” refers to the administration of at least two agents or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent.

As used herein, the term “pharmaceutical composition” refers to the combination of an active agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

As used herein, the term “treating” includes reducing or alleviating at least one adverse effect or symptom of a disease or disorder through introducing in any way a therapeutic composition of the present technology into or onto the body of a subject. “Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present technology.

### Embodiments of the Technology

Provided herein is technology relating to compositions containing bioactive non-methylene interrupted fatty acids (NMIFAs) for use in improving skin barrier function and related diseases and disorders. Below, sources of NMIFAs, compositions and formulations comprising these agents, and methods for making the compositions and formulations and uses of the compositions and formulations are described.

The skin barrier layer has the most important role in maintaining skin moisture balance. The barrier layer/extracellular domain of the stratum comeum is composed of lipids, predominately ceramides, free fatty acids, and cholesterol, in a precise lipid lamellar organization. See Bouwstra J, Pilgram G, Gooris G, Koerten H, Ponec M. New aspects of the skin barrier organization. Skin Pharmacol Physiol. 2001; 14(Suppl. 1):52-62; Oh M J, Cho Y H, Cha S Y, Lee E O, Kim J W, Kim S K, et al. Novel phytoceramides containing fatty acids of diverse chain lengths are better than a single C18-ceramide N-stearoyl phytosphingosine to improve the physiological properties of human stratum corneum. Clin Cosmet Investig Dermatol. 2017; 10:363-71. Sodium lauryl sulfate (SLS; also known as sodium dodecyl sulfate) is a type of concentrated soap/surfactant/detergent with an anionic hydrophilic polar group. See Casari A, Fametani F, De Pace B, Losi A, Pittet J C, Pellacani G, et al. In vivo assessment of cytological changes by means of reflectance confocal microscopy-demonstration of the effect of topical vitamin E on skin irritation caused by sodium lauryl sulfate. Contact Derm. 2017; 76(3):131-37. SLS is used in acute- and cumulative irritation skin barrier injury models; and in acute irritant contact dermatitis- and inflammation models. See Lee C H, Maibach H I. The sodium lauryl sulfate model: an overview. Contact Derm. 1995; 33(1):1-7. SLS was chosen as an irritant because of its ability to penetrate and impair the skin barrier. See Agner T. Noninvasive measuring methods for the investigation of irritant patch test reactions. A study of patients with hand eczema, atopic dermatitis and controls. Acta Derm Venereol Suppl (Stockh). 1992; 173:1-26. SLS is typically applied once at 1-2% for 24 hours (2% for 24 h was used herein); or repeatedly at lower concentrations (e.g., 0.1%) over 3 weeks. See De Jongh C M, Verberk M M, Withagen C E, Jacobs J J, Rustemeyer T, Kezic S. Stratum corneum cytokines and skin irritation response to sodium lauryl sulfate. Contact Derm. 2006; 54(6):325-33. At 2%, SLS can induce localized hyperalgesia and inflammation, and release pro-inflammatory pain mediators; inflammation can last up to 6 days during the irritation period. See, Petersen U. Lyngholm A M, Arendt-Nielsen L. A novel model of inflammatory pain in human skin involving topical application of sodium lauryl sulfate. Inflamm Res. 2010; 59(9):775-81.

Transepidermal water loss (TEWL) is the most important objective measurement for assessing barrier function and skin integrity in healthy individuals, and those with skin diseases associated with skin barrier dysfunction. TEWL is measured noninvasively with an evaporimeter probe to detect the quantity of low-level condensed water diffusing/transpiring across a fixed area of stratum corneum skin surface per unit time. See Akdeniz M, Gabriel S, Lichterfeld-Kottner A, Blume-Peytavi U, Kottner J. Transepidermal water loss in healthy adults: a systematic review and meta-analysis update. Br J Dermatol. 2018; 179(5):1049-55; Alexander H, Brown S, Danby S, Flohr C. Research techniques made simple: transepidermal water loss measurement as a research tool. J Invest Dermatol. 2018; 138(11):2295-300. Higher TEWL is associated with skin barrier impairments and damaged and irritated stratum corneum and epidermis, as seen in unhealed wounds, in skin scarring, in response to food allergens, and in psoriasis, eczema, and particularly atopic dermatitis. See Montero-Vilchez T, Segura-Fernandez-Nogueras M V, Perez-Rodriguez I, Soler-Gongora M, Martinez-Lopez A, Fernandez-Gonzalez A, et al. Skin barrier function in psoriasis and atopic dermatitis: transepidermal water loss and temperature as useful tools to assess disease severity. J Clin Med. 2021; 10(2):359. Lower TEWL is associated with healthy skin, and improved skin barrier-function and structure. A reduction in TEWL generally indicates proportionately higher film (barrier) integrity and/or wound closure in wound healing studies. We assessed SLS-induced changes to skin by measuring TEWL.

Damage to the stratum corneum outer skin layer can lead to increased surface water. This was measured noninvasively by surface impedance as electroconductivity, an indirect measure of the retained water content of the skin as a function of the skin's dielectric value. Skin impedance can be measured alongside TEWL to compare the two techniques and gain additional confidence in results [17, 23-25]. See Agner, supra; KaliaYN, Guy R H. The electrical characteristics of human skin in vivo. Pharm Res. 1995; 12(11):1605-13; Nicander I, Rantanen I, Rozell B L, Soderling E, Ollmar S. The ability of betaine to reduce the irritating effects of detergents assessed visually, histologically and by bioengineering methods. Skin Res Technol. 2003; 9(1):50-8; Sim D, Kim S M, Kim S S, Doh I. Portable skin analyzers with simultaneous measurements of transepidermal water loss, skin conductance and skin hardness. Sensors. 2019; 19(18):3857.

Visual assessment of skin reactions and color has long been used to evaluate the safety of chemicals and preparations that contact skin. See Farage M A, Maibach H I, Andersen K E, Lachapelle J M, Kern P, Ryan C, et al. Historical perspective on the use of visual grading scales in evaluating skin irritation and sensitization. Contact Derm. 2011; 65(2):65-75. Skin redness was evaluated by Visual Expert Grading, on a scoring scale of 1-10, following high resolution photography of test sites.

The results presented herein represent the first time SA (here, as a 24% w/w SA fatty acid composition) was formally tested in any controlled clinical setting, for topical or oral use. The primary goal of this first pilot study was to determine if NMIFA treatment could improve irritant-induced barrier damage relative to damaged skin without any other treatment. Specifically, our pilot study with 7 subjects was conducted to test if an NMIFA composition could reduce TEWL, impedance, and redness in the well-established SLS irritation injury model. We also report results on stability and repeat insult patch test safety studies, which were clinical prerequisites for conducting the SLS study.

### Non-Methylene-Interrupted Fatty Acids

The term non-methylene-interrupted fatty acid, the acronym for which is NMIFA, refers to a fatty acid with a series of double bonds in which at least one adjacent pair of double bonds is separated by at least two carbon atoms, i.e., by a group other than a single methylene group. Examples of NMIFA include, but are not limited to, 5,11,14-eicosatrienoic acid (sciadonic acid); 5,9,12-cis-octadecatrienoic acid (pinolenic acid); and 5,11,14,17-eicosatetraenoic acid (juniperonic acid). Preferred NMIFAs have the following formula, wherein the NMIFA is an acid, a salt or an ester, and R1 is a C₁-C₅ alkyl group and R2 is a C₂-C₆ alkyl group, may be advantageously used for the preparation of a composition intended to modulate the metabolism of lipids in superficial mammalian tissues.

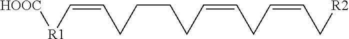

Particularly preferred NMIFAs are those in which R1 is a C₃ alkyl group and R2 is a C₂-C₆ alkyl group, or in which R2 is a C₄ alkyl group and R1 is a C₁-C₅ alkyl group. The most preferred is that in which R1 is an n-propyl group and R2 is an n-butyl group (5,11,14-eicosatrienoic acid, also called 20:3(5,11,14)). The NMIFAs may be preferably provided as triglycerides, phospholipids, fatty acids ester, free fatty acids or combinations thereof.

Sciadonic acid (5,11,14-eicosatrienoic acid, 20:3Δ5,11,14) is a polyunsaturated fatty acid containing non-methylene-interrupted double bonds, such as a Δ5-ethylenic bond. Sciadonic acid is often found in gymnosperms, in seed oils, leaves, and wood. It is also found in a few angiospernns, especially in seed oils. Sciadonic acid has several biological activities, including lowering triglyceride and cholesterol levels, reducing reperfusion injury, modifying autoimmune response, having cannabimimetic effect, treatment of skin disease, and treatment of sensitive or dry skin. WO 95/17987 (The Regents of the University of California) shows that broad class of NMIFAs, including 5,11,14-eicosatrienoic acid, may be used in an effective amount for suppressing autoimmune diseases in general, for example rheumatoid arthritis, lupus erythmatosis, multiple sclerosis, myasthenia gravis, and about 30 other diseases currently known. NMIFAs, including 5,11,14-eicosatrienoic acid, are further described in U.S. Pat. Nos. 5,456,912 and 6,280,755 as well as US Publ. No. 20120156171, each of which is incorporated herein by reference in its entirety.

Pinolenic acid ((5Z,9Z,12Z)-octadeca-5,9,12-trienoic acid; all-cis-5,9,12-18:3) is a fatty acid contained in Siberian Pine nuts, Korean Pine nuts and the seeds of other pines (Pinus species). The highest percentage of pinolenic acid is found in Siberian pine nuts and the oil produced from them. JP 61 058 536 (Nippon Oil) discloses a method for purifying pine nut oil containing at least 10% by weight of 5,9,12-cis-octadecatrienoic acid which exhibits a curative effect against arterial hypertension. WO 96 05 164 (Broadben Nominees Pty) discloses an anti-inflammatory preparation comprising a purified active fraction, for example 5,11,14,17-eicosatetraenoic acid, isolated from a lipid extract of Perna canalicullus or Mytilus edulis. Dihomopinoleic acid also finds use in the compositions of the present invention.

Some of the NMIFAs of the invention are naturally occurring substances. Others may be synthesized according to well-known published methodology (see, e.g., Evans et al., Chem. Phys. Lipids, 38, 327-342, 1995).

For example, 20:3(5,11,14) is a naturally occurring substance which generally occurs as one fatty acid in a mixture of fatty acids. This NMIFA is found in a wide variety of plants as minor or major fraction of the total fatty acid composition. Both the extraction of the mixture of fatty acid from their natural sources and the extraction of the 20:3(5,11,14) from the resulting fatty acids can be achieved by conventional extraction and purification methods well known among those skilled in the art.

The natural sources of fatty acids containing 20:3(5,11,14) are primarily plant seeds, and prominent among these are conifers and ornamental shrubs. The seed oils from these plants are similar to normal edible oils, containing largely oleic, linoleic and linolenic acids, but also containing useful amounts of NMIFAs. The following table lists examples of seeds whose lipid contents contain significant amounts of 20:3(5,11,14).

| | % of | | % of |
|---|---|---|---|
| | 20:3 (5, 11, 14) | | 20:3 (5, 11, 14) |
| | among total | | among total |
| Source | fatty acids | Source | fatty acids |
| J [symbol] | 14.8 | Sciadopi [symbol] | 15 |
| virgi [symbol] | | verticallata | |
| [symbol] | 3 | Ca [symbol] | 23 |
| [symbol] | | p [symbol] | |
| J [symbol] | 12.3 | C [symbol] | 14 |
| ch [symbol] | | rh [symbol] | |
| Torreya | 7 | Mo [symbol] | 7 |
| nucifera | | alp [symbol] | |
| [symbol] | 24 | Eph [symbol] | 22 |
| [symbol] | | [symbol] | |
| [symbol] | 10 | [symbol] | 6 |

| | % of | | % of |
|---|---|---|---|
| | 20:3 (5, 11, 14) | | 20:3 (5, 11, 14) |
| | among total | | among total |
| Source | fatty acids | Source | fatty acids |
| [symbol] | | lev [symbol] | |
| C [symbol] | 6 | [symbol] | 6 |
| [symbol] | | [symbol] | |
| G [symbol] | 2.2 | P [symbol] | 7 |
| [symbol] | | [symbol] | |

*see the Japanese patent JP [symbol] ( [symbol] ) [symbol]
indicates data missing or illegible when filed

Purification of 20:3(5,11,14) may be in particular achieved by (1) choosing a starting seed source high in total fat content and 20:3(5,11,14) content but not containing other contaminating trienes, in particular alpha-linolenic acid (18:3n-3) and gamma-linolenic acid (18:3n-6) (Podocarpus nagi is such an example); (2) extracting the lipids with isopropanol and chloroform according to the method of Nichols (Biochim. Biophys Acta 70: 417, 1963); (3) conventional degumming and decoloring methods; (4) preparing methyl esters with 2% methanolic sulfuric acid according to the method of Christie (p. 52-53, in Lipid Analysis, Pergamon Press, Oxford, 1982); (5) eluting 20:3(5,11,14) methyl ester from a silver nitrate impregnated acid-washed Florisil column with a hexane:ether mixture ranging from 9:1 to 8:2 (volume/volume) according to Carroll, J. Am. Oil Chem. Soc. 40: 413, 1963; Wilner, Chem. Ind (Lond) October, 30: 1839, 1965; Merck ChromNews 4(1): 1995; Anderson, J. Lipid Res. 6: 577, 1965; and Teshima, Bull. Jap. Soc. Scien. Fish. 44: 927, 1978); (6) removing contaminating silver ions by the method of Akesson (Eur J. Biochem. 9:463, 1969); and (7) optionally converting the methyl ester back to the free acid form by saponification in 1 M potassium hydroxide in 95% ethanol according to Christie (p. 51-52, in Lipid Analysis, Pergamon Press, Oxford, 1982).

In some preferred embodiments, the extracted oil comprises or consists essentially of triglycerides. In some embodiments, the weight percent of sciadonic acid expressed as grams per 100 grams fatty acids in the oil is from about 5% to 40%, 5% to 30%, 5% to 25%, 10% to 40%, 10% to 30%, or 10% to 25%.

In some preferred embodiments, the extracted oil comprises or consists essentially of free fatty acids. In some embodiments, the weight percent of sciadonic acid expressed as grams per 100 grams fatty acids in the free fatty acid composition is from about 5% to 40%, 5% to 30%, 5% to 25%, 10% to 40%, 10% to 30%, or 10% to 25%.

In some preferred embodiments, the extracted oil comprises or consists essentially of a combination of triglycerides and free fatty acids. In some embodiments, the weight percent of sciadonic acid expressed as grams per 100 grams fatty acids in the free fatty acid composition is from about 5% to 40%, 5% to 30%, 5% to 25%, 10% to 40%, 10% to 30%, or 10% to 25%.

In some preferred embodiments, the extracted oil comprises or consists essentially of triglycerides. In some embodiments, the weight percent of sciadonic acid expressed as weight of sciadonic acid per total weight of the composition (w/w) is from about 5% to 40%, 5% to 30%, 5% to 25%, 10% to 40%, 10% to 30%, or 10% to 25%.

In some preferred embodiments, the extracted oil comprises or consists essentially of free fatty acids. In some embodiments, the weight percent of sciadonic acid expressed as weight of sciadonic acid per total weight of the composition (w/w) is from about 5% to 40%, 5% to 30%, 5% to 25%, 10% to 40%, 10% to 30%, or 10% to 25%.

In some preferred embodiments, the extracted oil comprises or consists essentially of a combination of triglycerides and free fatty acids. In some embodiments, the weight percent of sciadonic acid expressed as weight of sciadonic acid per total weight of the composition (w/w) is from about 5% to 40%, 5% to 30%, 5% to 25%, 10% to 40%, 10% to 30%, or 10% to 25%.

### Bioactive Compositions and Formulations

The present invention provides bioactive compositions and formulations comprising one or more bioactive NMIFAs.

In preferred embodiments, the compositions and formulations of the present invention have a content of at least 1%, 2%, 5%, 10%, 20%, 30% or 40% NMIFAs on a w/w basis (total weight of NMIFA/total weight of the formulation), or from about 1% to 40%, 2% to 40%, 5% to 40%, 10% to 40%, 1% to 30%, 2% to 30%, 5% to 30%, 10% to 30%, 1% to 25%, 2% to 25%, 5% to 25%, or 10% to 25% NMIFAs on w/w basis. Preferred NMIFAs include, but are not limited to, sciadonic acid, juniperonic acid, pinoleic acid, dihomopinoleic acid, coniferonic acid (5, 9, 12, 15 18:4), and synthetic fatty acids selected from the group consisting of 1, 11, 14, 17 20:4; 2, 11, 14, 17 20:4; 3, 11, 14 17 20:4; 4, 11, 14 17 20:4; 6, 11, 14 17 20:4; 7, 11, 14 17 20:4; 1, 9, 12, 15 18:4; 2, 9, 12, 15 18:4; 3, 9, 12, 15 18:4; 4, 9, 12, 15 18:4, 5, 9, 12, 15 18:4, 1, 11, 14 20:3; 2, 11, 14 20:3; 3, 11, 14 20:3; 4, 11, 14 20:3; 6, 11, 14 20:3; 1, 9, 12 18:3; 2, 9, 12 18:3; 3, 9, 12 18:3; and 4, 9, 12 18:3 fatty acids.

It will be understood that the fatty acids may be provided in the formulation as free fatty acids, as ethyl esters, or in the form of diglycerides, triglycerides, or phospholipids to which the fatty acid is attached. The formulations are preferably characterized by comprising a particular ratio of the bioactive fatty acids to one another or as having a defined weight/weight (w/w) percentage of the bioactive fatty acids which refers to the weight of the specific fatty acid per total weight of fatty acids in the formulation (i.e., grams the specified acid per 100 grams of fatty acids in the lipid formulation).

Thus, the formulations according to the present technology include fatty acids analogous to naturally occurring fatty acids, especially NMIFAs or their analogs, alone in combination with other short and/or medium chain fatty acids, or naturally occurring lipids comprising the fatty acids. Incorporation of the fatty acids in naturally occurring lipids (e.g., monoglycerides, diglycerides, triglycerides, and/or phospholipids) produces a compound with different absorption characteristics compared to free fatty acids. In addition, it is contemplated that incorporating fatty acids in naturally occurring lipids (e.g., monoglycerides, diglycerides, triglycerides, and/or phospholipids) may also increase the bioavailability or stability.

In some embodiments, the fatty acids in the formulation are esterified to a triglyceride, diglyceride, monoglyceride or phospholipid molecule. In some embodiments, the fatty acids in the lipid formulation are provided as ethyl esters. In some embodiments, structured esters or lipids are provided such as structured esters, diglycerides, triglycerides or phospholipids wherein at least one ester linkage in the structured ester, diglyceride, triglyceride or phospholipid contains a short chain or medium chain fatty acid and least one other ester linkage in the structured ester, diglyceride, triglyceride or phospholipid contains an NMIFA.

In some embodiments, the fatty acids in the formulations are provided by blending one or more oils or lipids. In some embodiments, the NMIFAs are provided from one or more natural sources as described above. In some preferred embodiments, the NFIMAs are formulated with one or more carriers or excipients that do not naturally occur with the NMIFAs.

In some embodiments, the formulations are suitable for human consumption on a daily basis for an extended period of time, e.g., 1 month, 2 months, 6 months, 1 year or 2 years, when provided in daily dosage of from 200 mg to 5 or 10 grams. In some embodiments, the lipid formulations further comprise a food safe antioxidant. In some embodiments, the lipid formulations are provided in an oral delivery vehicle, food product, nutritional supplement, dietary supplement or functional food.

The present invention likewise provides methods of using the formulations. These methods and uses are described in detail below but may be summarized as follows. In some embodiments, the present invention provides methods of treating a subject suffering from inflammation comprising administering to the subject the bioactive lipid and non-fatty acid ant-inflammatory drug formulation/s or oral delivery vehicle/s, food product, nutritional supplement, dietary supplement or functional food comprising the lipid formulation to a subject in need thereof. In some embodiments, the administration is oral, topical, parenteral, enteral, transdermal, intradermal, intraocular, intravitreal, sublingual, or intravaginal and may preferably comprise an effective amount of the composition.

Provided herein are pharmaceutical formulations comprising a therapeutically effective amount of a composition according to the present technology and a pharmaceutically acceptable carrier, diluent, or excipient (including combinations thereof).

A composition according to the technology comprises or consists of a therapeutically effective amount of a pharmaceutically active agent. In some embodiments, it includes a pharmaceutically acceptable carrier, diluent, or excipient (including combinations thereof). Acceptable carriers or diluents for therapeutic use are well known in the pharmaceutical art and are described, for example, in Remington's Pharmaceutical Sciences, Mack Publishing Co. (A. R. Gennaro edit. 1985). The choice of pharmaceutical carrier, excipient, or diluent is selected with regard to the intended route of administration and standard pharmaceutical practice. The pharmaceutical comprise as, or in addition to, the carrier, excipient, or diluent any suitable binder(s), lubricant(s), suspending agent(s), coating agent(s), and/or solubilizing agent(s).

This pharmaceutical composition will desirably be provided in a sterile form. It may be provided in unit dosage form and will generally be provided in a sealed container. A plurality of unit dosage forms may be provided.

Pharmaceutical compositions within the scope of the present technology may include one or more of the following: preserving agents, solubilizing agents, stabilizing agents, wetting agents, emulsifiers, sweeteners, colorants, flavoring agents, odorants, and/or salts.

Compounds of the present technology may themselves be provided in the form of a pharmaceutically acceptable salt. In addition, embodiments may comprise buffers, coating agents, antioxidants, suspending agents, adjuvants, excipients, and/or diluents. Examples of preservatives include sodium benzoate, sorbic acid, and esters of p-hydroxybenzoic acid.

They may also contain other therapeutically active agents in addition to compounds of the present technology. Where two or more therapeutic agents are used they may be administered separately (e.g., at different times and/or via different routes) and therefore do not always need to be present in a single composition. Thus, combination therapy is within the scope of the present technology.

The routes for administration (delivery) include, but are not limited to, one or more of: oral (e.g. as a tablet, capsule, or as an ingestible solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, via the penis, vaginal, epidural, sublingual.

It is to be understood that not all of the agent need be administered by the same route. Likewise, if the composition comprises more than one active component, then those components may be administered by different routes.

If the agent of the present technology is administered parenterally, then examples of such administration include one or more of: intravenously, intra-arterially, intraperitoneally, intrathecally, intraventricularly, intraurethrally, intrastemally, intracranially, intramuscularly, or subcutaneously administering the agent; and/or by using infusion techniques.

In some embodiments, pharmaceutical compositions adapted for oral administration are provided as capsules or tablets; as powders or granules; as solutions, syrups or suspensions (in aqueous or non-aqueous liquids); as edible foams or whips; or as emulsions. Tablets or hard gelatin capsules may comprise lactose, maize starch or derivatives thereof, stearic acid or salts thereof. Soft gelatin capsules may comprise vegetable oils, waxes, fats, semi-solid, or liquid polyols etc. Solutions and syrups may comprise water, polyols and sugars. For the preparation of suspensions, oils (e.g., vegetable oils) may be used to provide oil-in-water or water-in-oil suspensions. An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

Alternatively, the agent of the present technology can be administered in the form of a suppository or pessary, or it may be applied topically in the form of a gel, hydrogel, lotion, solution, cream, ointment or dusting powder. The agent of the present technology may also be dermally or transdermally administered, for example, by the use of a skin patch. They may also be administered by the pulmonary or rectal routes. They may also be administered by the ocular route. For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the agent of the present technology can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

If the agent of the present technology is administered parenterally, then examples of such administration include one or more of intravenously, intra-arterially, intraperitoneally, intrathecally, intraventricularly, intraurethrally, intrastemally, intracranially, intramuscularly or subcutaneously administering the agent; and/or by using infusion techniques.

For parenteral administration, the agent is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Typically, a physician will determine the actual dosage which will be most suitable for an individual subject. The specific dose level and frequency of dosage for any particular patient may be varied and will depend upon a variety of factors including the activity of the specific compound employed; the metabolic stability and length of action of that compound; the age, body weight, general health, sex, diet, mode and time of administration; rate of excretion; drug combination; the severity of the particular condition; and the individual undergoing therapy. The agent and/or the pharmaceutical composition of the present technology may be administered in accordance with a regimen of from 1 to 10 times per day, such as once or twice per day. For oral and parenteral administration to human patients, the daily dosage level of the agent may be in single or divided doses.

Depending upon the need, the agent may be administered at a dose of from 0.01 to 30 mg/kg body weight, such as from 0.1 to 10 mg/kg or from 0.1 to 1 mg/kg body weight. Naturally, the dosages mentioned herein are exemplary of the average case. There can, of course, be individual instances where higher or lower dosage ranges are merited.

“Therapeutically effective amount” refers to the amount of the therapeutic agent that is effective to achieve its intended purpose, i.e., reduction of inflammation and associated symptoms. While individual patient needs may vary, determination of optimal ranges for effective amounts of the compounds related to the technology is within the skill of the art. Generally, the dosage regimen for treating a condition with the compounds and/or compositions of this technology is selected in accordance with a variety of factors, including the type, age, weight, sex, diet and medical condition of the patient; the severity of the dysfunction; the route of administration; pharmacological considerations such as the activity, efficacy, pharmacokinetic and toxicology profiles of the particular compound used; whether a drug delivery system is used; and whether the compound is administered as part of a drug combination and can be adjusted by one skilled in the art. Thus, the dosage regimen actually employed may vary widely and therefore may deviate from the exemplary dosage regimens set forth herein.

### Use to Improve Skin Barrier Function and Treat Related Conditions

In some preferred embodiments, the present invention provides for use of the compositions and formulations described above to improve skin barrier functions and to treat conditions associated with loss of barrier function in human and animal subjects. In some preferred embodiments, the present invention provides NMIFA compositions for use in treating a disease or condition associated with loss of barrier function in a human or animal subject or to increase collagen production.

In some preferred embodiments, the disease or condition associated with loss of barrier function in a human or animal subject is selected from the group consisting of inflammation or irritation caused by an allergen, inflammation or irritation caused by a chemical irritant, inflammation or irritation caused by nickel, latex, skin disinfectants, topical skin corticosteroids and glucocorticoids, topical germicidal compositions, inflammation or irritation caused by plant toxins or mechanical plant irritants, inflammation or irritation caused by insect toxins, inflammation or irritation caused by exposure to an oil-in-water (O/W) emulsion, inflammation or irritation due sunburn or damage caused by UV-B exposure, inflammation or irritation due to exposure to a skin whitening, bleaching, or lightening agent, inflammation or irritation caused by environmental pollutant, skin surface dysbiosis-induced irritation, dry skin due to loss of skin moisture, loss of skin elasticity, skin wrinkling, and frown lines. It is contemplated that in some preferred embodiments, use of the compositions of the present invention can reduce or eliminate the need for use of topical steroids such as glucocorticoids, reduce or eliminate the need for topical non-steroidal medications and/or reduce or eliminate the use of other classes of topical drugs used to treat irritant-induced barrier dysfunction.

In some preferred embodiments, the allergen is pet dander. In some preferred embodiments, the chemical irritant is bezalkonium chloride (BAC), sodium lauryl sulfate (SLS), benzoyl peroxide, a PABA-based chemical, an adhesive, a chemical fragrance, a chemical found in household cleaners (e.g., dish detergents, laundry detergent, window cleaners, furniture polish, drain cleaners and toilet disinfectants), oxybenzones, benzophenones, cinnamates, dibenzoylmethanes, chlorine, N,N-Diethyl-meta-toluamide (DEET), residual solvent (e.g., xylene, toluene, and trimethylbenzene, octanoic acid, nonanoic acid, decanoic acid, and n-propanol and other alcohols) or a soap. In some preferred embodiments, the mechanical plant irritant is a plant thorn, plant spine, plant nettle, plant glochid, plant trichome, or sharp-edged leaves). In some preferred embodiments, the plant toxin is urushiol (such as found in poison ivy, poison oak and poison sumac), calcium oxalate, protoanemonin, isothiocyanates, bromelain, diterpene esters, alkaloids, and other chemical irritants such as naphthoquinone or other plant-derived acids. In some preferred embodiments, the insect toxin is formic acid or mosquito saliva. In some preferred embodiments, the O/W emulsion is Aqueous Cream BP 2001, Clioquinol Cream BP 1999 without clioquinol, Nonionic Hydrophilic Cream DAB 2001 without glycerol, Hydrophilic Skin Emulsion Base NRF S. 25., point of time 2001, without glycerol, and Base Cream DAC In some preferred embodiments, the kin whitening, bleaching, and lightening agent is magnesium-1-ascorbyl-2-phosphate (MAP), hydroxyanisole, N-acetyl-4-S-cysteaminylphenol, arbutin (hydroquinone-beta-d-glucopyranoside) or hydroquinone. In some preferred embodiments, the environmental pollutant is solar ultraviolet radiation (UVR), a polycyclic aromatic hydrocarbon (PAH), a volatile organic compounds (VOC), nitrogen oxides (NOx), particulate matter (PM), and cigarette smoke.

In some embodiments, the irritation is skin surface dysbiosis-induced irritation. In some embodiments, the skin surface dysbiosis-induced irritation is caused by a skin pathogen or disruption of the skin microbiome. By reducing inflammation and altering lipid composition of both host and microbial species, it is contemplated that SA will normalize the skin microbiome. This in turn, improves skin barrier dysfunction. The skin microbiota is an integral part of the skin barrier. It protects the host from pathogens by competing for nutrients and space. Some produce antimicrobial compounds, which block the growth of competitors. Symbionts also alter the skin barrier via bacterial enzymes, such as proteases that impact comeocyte desquamation, or lipases, which break down skin surface lipids. Staphylococcus aureus colonization is found in up to 90% of atopic dermatitis patients, a stereotypical disease of skin barrier dysfunction. It produces ceramidase, which breaks down ceramides, an essential component of the skin barrier. S. aureus also produces ca toxins that impede wound healing and bring epithelial barrier disintegration. Scabies mites (Sarcoptes scabiei) alter the skin microbiota by breaching the physical barrier. Epidemiologic studies in scabies patients confirmed secondary bacterial infections by two clinically important pathogens S. aureus and Streptococcus pyogenes. There has also been a growing awareness of fungi and their interaction with the skin barrier. When the chemical composition (i.e., sweat, pH) of the host epidermis is disturbed, Malassezia spp. acquire pathogenicity and liberate an array of bioactive indoles, lipases, and phospholipases. These molecules further modify the function of the skin barrier. Epithelial barrier disruption can lead to a vicious itch-scratch cycle, which leads to skin barrier dysfunction.

### Experimental

### 1. Methods

### Test Materials all Studies

The starting plant seed source was from Nageia nagi seeds (China), used for preparation of our commercial Delta-5® oil currently sold by SciaEssentials® (INCI name: Nageia nagi Seed Oil). Seeds were sun-dried, tempered, and cracked to partially remove the hulls and skins surrounding the nut “meat”. This material was pressed, filtered, then refined, bleached, deodorized, and winterized (RBDW) to obtain the final cosmetic-grade oil. The oil was supplemented with mixed natural tocopherols (Dadex GT-2 NGM—Food, Mississauga, ON, Canada) to a final concentration of 1500 mg Dadex GT-2/kg oil, then stored under nitrogen at −20° C. in 10 mL brown dropper bottles until use. Main fatty acids present (as % total fatty acid methyl ester peaks identified by gas chromatography) were 15.8% oleic acid (18:1n-9), 42.3% linoleic acid (18:2n6), 11.4% eicosadienoic acid (dihommolinoleic acid; 20:2n-6); and 24.4% SA. The fatty acids are present almost exclusively bound to glycerol as triacylglycerols based on our analyses and published literature. Oil was of high quality for skin applications based on color lack of odor, and low viscosity, with 0.110% free fatty acids and peroxide value of 0.66 mEq/kg. Levels of microbes (Salmonella, E. Coli, and yeast and mold) and heavy metals (arsenic, cadmium, mercury, lead) were negative or within acceptable limits. Oil extractions and refinement, and analytical testing were performed by our commercial partners using accredited laboratories and procedures. Prior to experimentation, oils were thawed at room temperature.

### Stability Testing Study Methods and Results

Prior to safety and clinical testing, stability testing was performed. Open vessels containing three different lots of Delta-5 oil were placed in a controlled temperature chamber at 25- and 40° C. for 3 months, with assessments for changes in color, odor (due to fatty acid oxidation), and homogeneity of appearance (due to precipitation). Oils were evaluated initially, and at weeks 1, 2, 3, 4, 8, and 12. At both temperatures, and at all time points, there were no changes observed in color and odor; and the oils remained homogeneous (pictures not shown). For personal care testing, 3 months stability at 40° C. is generally equated with 1 year stability at 25° C. For an oil in a neat formulation with only exogenous tocopherols added as an additional antioxidant, and up to 78% polyenes (dienes and trienes), this represents very good stability in an open system, not nitrogen gas-blanketed to prevent lipid oxidation. In our SA-rich oils kept at −20° C. under nitrogen, we have not observed changes in fatty acid content nor peroxide value after 3 years storage. As described in the Introduction section, although SA is a triene, its oxidative stability is more like that of a more stable diene, due to the non-methylene interrupted arrangement of the three double bonds. This factor is likely responsible for the prolonged stability observed in this preliminary stability investigation

### Repeat Insult Patch Test Study Methods and Results

A standard Repeat Insult Patch Test (RIPT) was conducted to verify Delta-5® oil was not a contact sensitizer nor skin irritant. See Association of Food and Drug Officials of the United States. Appraisal of the Safety of Chemicals in Food, Drugs and Cosmetics. 1965. The study was conducted from 4/5-5/12/21 at Advanced Science Laboratories, Inc. (New City, NY) following Standard Operating Procedures with modifications for our non-viscous oil, in compliance with Institutional Review Board guidelines (CFR Title 21 Part 56, Subparts A, B, C, D). Evaluators were required to pass a visual discrimination examination overseen by a Board-Certified Ophthalmologist using the Farnsworth-Munsell 100 Hue Test, which determines a person's ability to discern color against a black background. This test was additionally modified to include a flesh tone background approaching actual use conditions; erythematous skin was graded according to intensity.

### Inclusion and Exclusion Criteria for RIPT

Inclusion criteria consisted of being free of any dermatological or systemic disorder; free of any acute or chronic disease; in general good health; willing to complete a preliminary medical history; willing to sign an informed consent; willing to cooperate with the research team; willing to have test materials applied; and willing to complete the full course of the study. Exclusion criteria consisted of being under 18 years of age; currently taking topical or systemic medication that could mask or interfere with test results; having a history of acute or chronic disease that might interfere with or increase risk associated with study participation; having chronic skin allergies; and being pregnant or lactating.

### Recruitment and Subjects for RIPT

Participants were recruited by advertisements in local periodicals, community bulletin boards, phone solicitation, electronic media, or combinations thereof. There were 63 subjects enrolled, and 53 completers. This represents a typical percentage (84%) of completers for RIPT testing, which involves multiple patch applications and site visits for scoring. Dropouts resulted from subjects being unreliable, developing COVID-19, and for other reasons, unrelated to Delta-5® per se. The age range was 19-74, with 15 males, and 39 females with racial and ethnic backgrounds.

### Testing Equipment for RIPT

Equipment consisted of 2 cm×2 cm gauze patches, covered with occlusive tape (the assemblage was custom made by Strukmyer, LLC, Mesquite, TX); and 1 mL volumetric syringes without needles to dispense Delta-5®. Subjects were requested to bathe or wash as usual before arrival at the facility. Delta-5® was refrigerated, then prior to use, equilibrated at room temperature. Delta-5® (0.2 mL) was dispensed onto patches, and patches applied directly to infrascapular regions of the back, to right or left of midline. Subjects were instructed to not wet or expose test areas to direct sunlight. After 24 h, patches were removed by panelists at home, and fresh patches with Delta-5® re-applied at home. This procedure was repeated for at least nine 24-h induction phase exposures.

### Scoring and Results for RIPT

Reactions were scored just before applications 2-9; and at the re-test date following application 9 at 24- and 48 h after patch removal. Comparisons were made between the 9 inductive responses and the retest doses. At the study conclusion, a consulting Dermatologist confirmed the Study Director's conclusions. The scoring scale ranged from 0.0 (no evidence of any effect) to 4.0 (severe deep red erythema with vesiculation or weeping), with intermediary scores with defined characteristics of 0.5, 1.0, 2.0, and 3.0. Amongst the 63 subjects initially enrolled and the 53 completers, evaluators scored all sites as 0.0, indicating Delta-5® was not a contact irritant nor skin sensitizer. There were also no observed adverse reactions demonstrating erythema and/or edema. Having demonstrated good stability, and lack of irritancy and skin sensitization, we proceeded to our skin irritation barrier function testing, described below.

### Transepidermal Water Loss and Impedance Study Methods and Skin Redness Methods

The study was conducted at Advanced Science Laboratories (New City, NY) following Standard Operating Procedures with modifications for our non-viscous Delta-5® oil, in compliance with Institutional Review Board guidelines (CFR Title 21 Part 56, Subparts A, B, C, D).

### Inclusion and Exclusion Criteria; Recruitment and Subjects

Inclusion and exclusion criteria, informed consent, and recruitment were as described for the Repeat Insult Patch Test. For TEWL testing, an additional inclusion criterion was that subjects abstain from using moisturizing products on the volar forearm, for at least two days prior to study commencement. There were 7 subjects enrolled, and 7 completers, monitored for 28 d, across three sites on the skin. The 7 subjects were coded, 56 0949, 48 9460, 39 1287, 68 2278, 70 9478, 56 3379, and 58 9750. The age range was 45-58; and all were female and Caucasian.

### Testing Equipment

Bioengineering parameters were assessed by TEWL and impedance. TEWL was measured with an open-chamber DermaLab System computerized Evaporimeter (cyberDERM, Inc., Cortex Technology, PA), equipped with a built-in analog-to-digital (A/D) converter for sending data streams to the host computer via a serial interface. TEWL readings were conducted in a quiet area, apart from general traffic and test center activity. Readings were obtained by placing the probe lightly in contact with the skin surface. Measurements of cutaneous evaporation rate are expressed as g/m²h. TEWL measurements require about 1 minute to allow for equilibrium in the chamber.

Skin surface water was measured by surface impedance as electroconductivity with a Novameter (Nova Dermal Phase Meter, Model DPM 9003, Nova, Technology Corp., Gloucester, MA). The meter provides a relatively, indirect measure of skin retained water content as a function of the skin's dielectric value (see novatechcorp.com/dpm.html for a more technical explanation). Skin impedance was recorded automatically when equilibrium was achieved, which is typically after 5 measurements in the same spot. Units are in arbitrary Dermal Phase Meter impedance units (DPMIU).

Skin redness was evaluated by colorimetric Visual Expert Grading (VEG) measurement from high resolution photography (photographs not shown), on a scoring scale of 0-10 (VEG Score; VEGS), utilizing equipment and techniques employed at Advanced Science Laboratories (formerly AMA Laboratories, Inc.; amalabs.com/services/claim-substantiation/matched-scientific). The camera does not touch the skin. A score of 0 represents no redness and clear skin; a score of 10 represents maximal redness possible. Redness scores were evaluated based on values at each time point, with—and without subtracting D0 and D1 values. Redness was also assessed by summing the counts across categorical scores 0, >0, 0-1, 2-4, and 5-7 (7—the highest score observed).

### Experimental Design

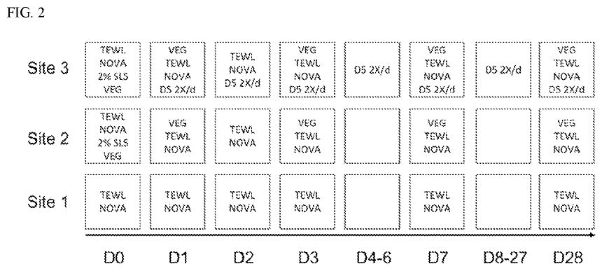

Panelists first reported to the testing facility with forearms devoid of topical treatments. Subjects were acclimated to ambient environment 15 min minimum prior to biophysical measurements. The acclimation procedure was repeated for each subsequent evaluation time point. All subjects received verbal instructions regarding product use and study restrictions. Subjects were required to apply 3 drops (about 27 mg/drop) of Delta-5® twice daily (162 mg total in the 6 drops/day). Each 3-drop application spread into a circle with diameter of 15-20 mm. Designated test site areas were midway between wrist and elbow; and left or fight inner volar forearm regions. Assignment of Delta-5® to left or right volar forearm was randomized. Test Site 1 served as untreated, undamaged control; Test Site 2 served as Untreated, SLS-damaged skin; Test Site 3 served as Delta-5® treatment applied to SLS-damaged skin. Delta-5® oil was received refrigerated, then prior to first use, equilibrated at room temperature, and kept at room temperature for the remainder of the experiment. The timing for measurements of TEWL, impedance and redness are explained below and in FIG. 2.

Day 0 (baseline): Baseline TEWL and impedance measurements were obtained from test sites 1-3 just before SLS-damage and initial Delta-5® application. The order of measurements was TEWL, impedance, then redness. Redness was measured on D0, but just after SLS was applied to Sites 2 and 3; and is designated D0*. SLS (0.2 g) was diluted to a 2% final concentration in distilled water, dispensed onto an occlusive, hypoallergenic patch and applied one time only directly to skin (Sites 2 and 3).

Day 1 (1 d after SLS): Patches were removed about 24 h post-application of 2% SLS and discarded. The order of measurement was redness (Sites 2-3), TEWL (Sites 1-3) and then impedance (Sites 1-3). After TEWL measurement, Delta-5® was applied to Site 3 twice daily each morning and before bedtime, from Day 1-Day 28.

Days 2 (i.e., 2 d after SLS, 1 d after Delta-5® oil): TEWL and impedance measured at Sites 1-3. Redness was not assessed. Delta-5® oil was applied to Site 3 twice daily on Days 1-28

Days 3, 7, and 28: Redness (Sites 2-3), TEWL (Sites 1-3) and then impedance (Sites 1-3) were measured. Delta-5® oil was applied to Site 3 twice daily on Days 1-28.

### Statistics

Data were first evaluated for normal distributions and equal variances. P-values generated from Shapiro-Wilk Test on TEWL and impedance differences between days (D2-D1, D3-D1, D7-D1, and D28-D1) were >0.01 indicating normal distribution, so data were evaluated with parametric testing with no data rank transformation. Effects of SLS alone (prior to application of Delta-5®) were evaluated by comparing D1-D0 with 1-tailed paired T tests (1-tailed since SLS is known a priori to increase TEWL and impedance). Technicians consistently applied Delta-5® to the more SLS-damaged site (Site 3); this was verified by evaluating D1-D0 TEWL values for sites to be treated with Delta-5® and untreated sites. Two-tailed T tests were used to test for temporal changes in the control site (no SLS). Following SLS damage, to determine if Delta-5® improved healing over the non-treated SLS sites, we evaluated differences between treated and untreated sites by evaluating D2−D1, D3−D1, D7−D1, and D28−D1, using repeated measures analysis of variance (ANOVA), with 1-tailed As there were changes in TEWL in the control group for D1-D0, a similar repeated measures ANOVA statistic was generated after adjustment for control values, by dividing values collected at each day by the corresponding control value. This correction resulted in a lower p-value for D3-D1, a time point at which there was more variability; variability was also reduced for other time points. Our main focus was thus on the control-corrected values for TEWL. Impedance values were evaluated as differences relative to D1, with and without correction for control values; results are shown without control-correction due to high variability in the control. For skin redness, similar statistical conclusions were reached with parametric T-testing and with Wilcoxon signed rank testing; so only parametric statistics are described for simplicity and consistency with the statistical approaches used for the TEWL and impedance evaluations. Redness was evaluated at each time point; and the data are also described as categorical variables. Statistics were mainly generated using SAS version 9.4 (SAS Institute, Inc., Cary, NC). Where appropriate, if not indicated otherwise, results in the text represent means±1 standard deviation (SD).

### 2. Results SLS Study

### TEWL-Control Skin and SLS Treatment Alone

TEWL increased in control skin as a function of time for unclear reasons. TEWL increased 23% from D0 to D1 (3.34 to 4.11 g/m²h), 34% from D0 to D3 (to 4.11 g/m²h) and 31% from D0 to D7 (to 4.37 g/m²h; <0.02 for these comparisons, 2-tailed T-tests). There were not significant differences in TEWL from D1 to D2, D1 to D3, D1 to D7, nor D1 to D28 (P>0.12, 2-tailed T-tests). Due to changes in control TEWL from baseline, subsequent statistics were adjusted for changes in time-matched control skin.

SLS profoundly increased TEWL. Combining pre-treated (sites to be treated with Delta-5®) and untreated SLS-damaged groups together (n=14), TEWL increased 7.8-fold from 3.47 at D0 to 27.19 g/m²h at D1 (p=0.0002). The p-value was ≤0.01, with groups separately (n=7). Technicians were instructed to apply Delta-5® to more damaged sites (Site 3). D1 TEWL was 57% higher in pre-treated sites than untreated sites (33.23 avg vs. 21.16 g/m²h, respectively; p<0.02).

### TEWL, Impedance-Delta-5 Effects

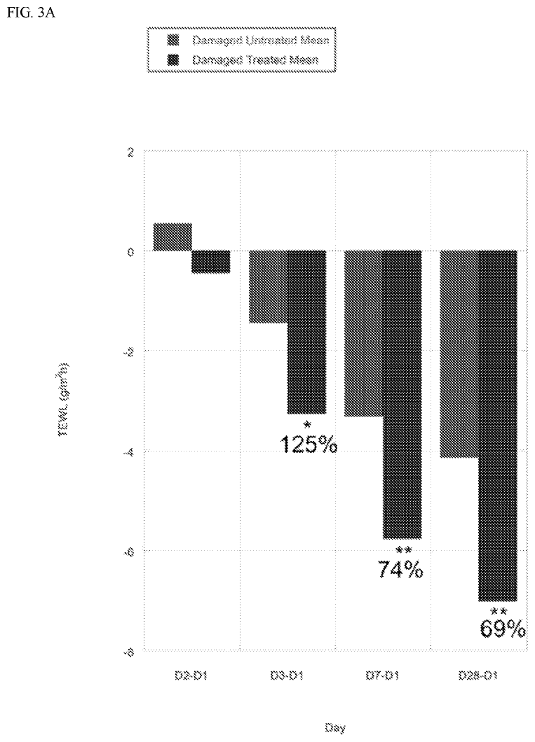

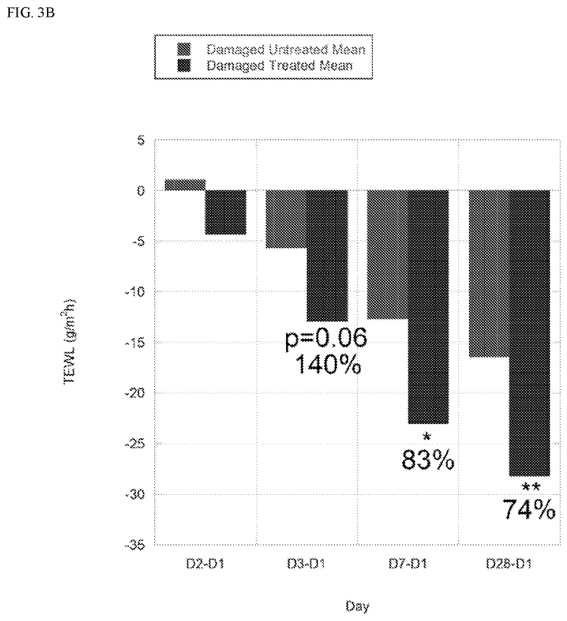

Relative to D1 TEWL (normalized by time-matched control values), Delta-5® decreased TEWL vs. untreated SLS sites, on D3 (125% more reduced; p=0.03), D7 (74% more reduced; p=0.01), and D28 (69% more reduced; p=0.006; Table 1; FIG. 3a). Differences were not significant for this statistic at D2 (means±SD were −0.5±4.1 for Delta-5-; and 0.5±3.2 g/m²h for untreated-SLS-damaged sites; p=0.23). Without normalization by time-matched control values, the same trends as above were observed relative to D1 values (p-values were 0.06, 0.02, and 0.01 for days 3, −7, and −28, respectfully; Table 1; FIG. 3b). With a small sample size of n=7, it is particularly important to evaluate individual subject responses. On D3, 71% of subjects (5 of 7) showed more improvement with Delta-5® than no treatment following SLS-damage. On days 7 and −28, 100% of subjects (all 7) showed more improvement.

Residual skin damage is the amount of damage from SLS remaining at each time point studied. This calculation is typically presented in TEWL experiments but is crude in that damage is assigned a value of 0 on D0 and 100% on Day 1 (after the irritant is applied). It was specifically calculated for untreated and treated sites as follows: (Dx/(D1−D0)) −(D0/(D1−D0)), where X=D2, D3, D7 and D28, D0 represents 0% damage, and D1 represents 100% damage. In untreated sites, residual damage was 102±98% (means±1 SD), 46±39, and 11±6% at days 3, 7, and 28, respectively. In treated sites, residual damage was 78%±52%, 33±25%, and 10 12% on these same days, respectively. Only on D7, was a slight statistical trend (p=0.09; 1-tailed, paired testing) observed for untreated and Delta-5® treated groups. By day 28, there was thus approximately 10% damage remaining (90% skin barrier repair) whether treated with Delta-5® or not, with this type of calculation.

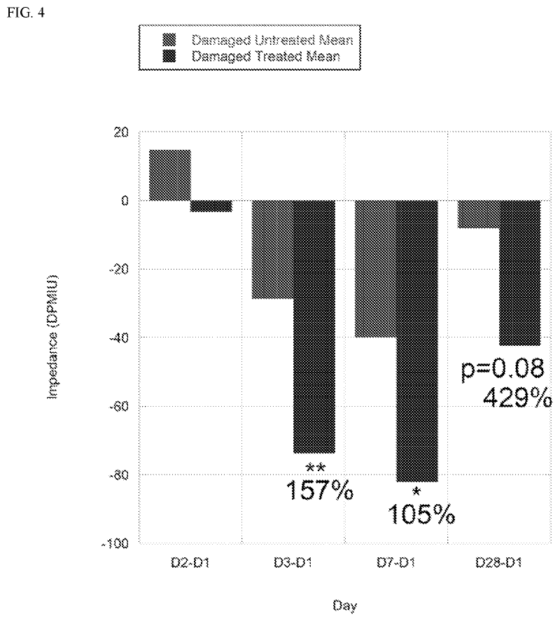

Skin surface impedance showed the same trends as TEWL. When TEWL was reduced, there was also less water on the skin surface and lower impedance. There were missing values for one subject (58 9750). This subject also had a clearly outlying impedance value for control undamaged skin on Day 1 (a value of 3.00 vs. a mean of 114.7±9.7 for the other 6 subjects) and was thus excluded. Skin impedance was not normalized to time matched controls as there was high variability in control values. This variability could be due to filling of superficial voids in the skin [28]. Relative to D1, Delta-5® decreased impedance vs. untreated SLS sites, on D3 (157% more reduced; p=0.01), D7 (105% more reduced; p=0.02), and D28 (429% more reduced; p=0.08; Table 1; FIG. 4). Relative to undamaged sites (D0), D1 DPMIU values were higher as expected, but statistical significance was not reached due to high variation (p=0.18 and p=0.26 for untreated- and treated damaged groups, respectively). By D2, impedance reached its maximum, being 47% higher than D0 (p=0.02-0.05); treated- and untreated SLS sites were not statistically significant from one another (p=0.26).

TABLE 1
TEWL and Impedance evaluated as differences from Day 1 in Delta-5 ®
oil-SLS damaged and untreated damaged groups at various time points.

| | Time points | | | |
|---|---|---|---|---|
| Means | D 2-D 1 | D 3-D 1 | D 7-D 1 | D 28-D 1 |
| | TEWL With Control Normalization | | | |
| Damaged | 0.54 ± 3.23 | −1.45 ± 3.37 | −3.32 ± 3.51 | −4.14 ± 3.47 |
| Untreated | | | | |
| Damaged Treated | −0.45 ± 4.09 | −3.26 ± 3.98* | −5.76 ± 4.47** | −7.02 ± 4.68** |
| P-Value | 0.24 | 0.03 | 0.01 | 0.01 |
| % difference | | 125.2 | 73.6 | 69.4 |
| | TEWL Without Control Normalization | | | |
| Damaged | 0.84 ± 10.42 | −5.14 ± 12.99 | −12.46 ± 13.61 | −15.86 ± 13.35 |
| Untreated | | | | |
| Damaged Treated | −4.53 ± 16.31 | −12.36 ± 17.35 | −22.80 ± 17.93* | −27.60 ± 18.52** |
| P-Value | 0.17 | 0.06^{Trend} | 0.02 | 0.01 |
| % difference | | 140.3 | 83.0 | 74.1 |
| | Impedance Without Control Normalization | | | |
| Damaged | 14.67 ± 84.85 | −28.67 ± 119.75 | −40.00 ± 118.30 | −8.00 ± 98.20 |
| Untreated | | | | |
| Damaged Treated | −3.33 ± 100.20 | −73.67 ± 148.65** | −82.00 ± 140.76* | −42.33 ± 145.19 |
| P-Value | 0.14 | 0.01 | 0.02 | 0.08^{Trend} |
| % difference | | 157.0 | 105.0 | 429.2 |

Values represent means for differences from each time point from day 1±1 SD for 6-7 subjects. Units for TEWL are g/m²h; units for impedance are in arbitrary Dermal Phase Meter impedance units (DPMIU). For impedance, subject 589750 was excluded due to missing values and an outlying value (n=6). Values from each time point were either normalized by the corresponding untreated control value for the corresponding time points, or not, as indicated. Asterisks are indicated as*p≤0.05; **p≤0.01, with p-values from 1-tailed, paired T-tests also provided (see Statistics section for details). Percents represent percent differences between treated- and untreated groups at each time point interval, and are provided when the p-value was between 0.05 and 0.10, as indicated as a statistical trend (“Trend”).

### Skin Redness

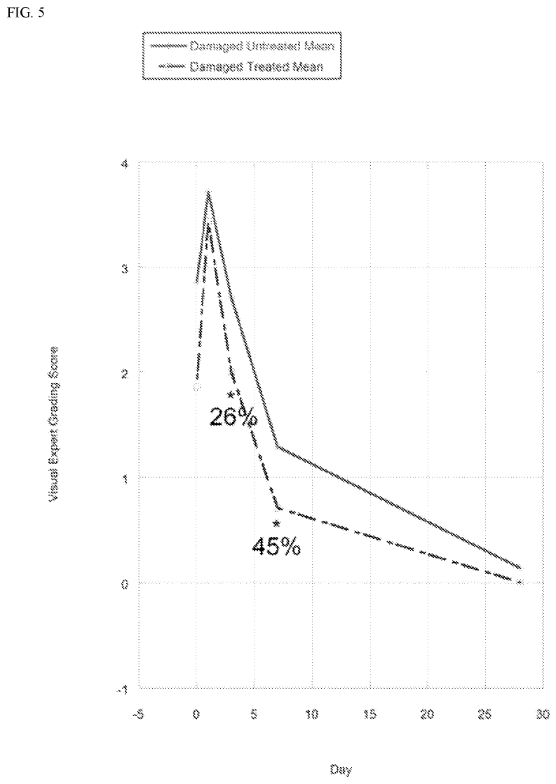

Redness was evaluated in untreated- and Delta-5®-treated sites after SLS application. Mean redness scores at each time point are shown in FIG. 5. Redness was also evaluated as a categorical variable by coming several redness scores together (Table 2). Redness VEGS ranged from 0-7 throughout the 28 days. Immediately after SLS application (D0*), 13/14 sites showed low levels of redness (VEGS≥1). D0*VEGS were 2.86±1.68 and 1.86±1.35 (means±SD) for untreated- and pre-treated sites, respectively. D0* means were not statistically significantly different from one another (p=0.14; 2-tailed t-test), with combined D0* value of 2.36±1.55 VEGS (14 sites). By D1, VEGS increased to 3.71±2.29 and 3.43±2.76 for untreated- and pre-treated sites, respectively, the maximal mean values observed for the time points assessed. D1 VEGS means were also not statistically significant from one another (p=0.36, 2-tailed t-test), with combined D1 VEGS of 3.57±2.44. D3 VEGS decreased to 2.71±1.98 and 2.00±1.91 for untreated- and treated sites, respectively; and D3-treated sites showed a statistically meaningful 26% reduction in redness vs. untreated sites (p=0.05, 1-tailed testing). On D3, highest redness category VEGS of 5-7 were observed in 29% of untreated- vs. only 14% of treated sites. On D3, all untreated sites showed some redness (VEGS>1); but 29% of treated sites were fully resolved (no redness; VEGS of 0). By D7, VEGS decreased further to 1.29±0.95 and 0.71±0.49 for untreated- and treated sites, respectively; and D7-treated sites showed a statistically meaningful 45% reduction in redness vs. untreated sites (p=0.05, 1-tailed testing). By D7, a VEGS of 5-7 was not observed in untreated-nor treated sites. However, on D7, 29% of untreated sites still had an intermediate VEGS of 2-4 vs. no 2-4 VEGS with treatment. Moreover, on D7, redness was fully resolved in 14% of untreated- vs. 29% of treated sites. By D28, redness was fully resolved in all untreated- and treated sites, excepting a VEGS of 1 at 1/14 sites. There were no statistically significant differences between untreated- and treated sites on D3 and D7 after subtracting D0*- or D1-starting values (i.e., D3-D0*, D3-D1, D7-D0*, D7-D1), due to large variance in the combined statistics.

TABLE 2
Redness categorical data evaluated in Delta-5 ® oil-
treated and untreated groups at various time points.

| | Days | | | | |
|---|---|---|---|---|---|
| Scores | D 0* | D 1 | D 3 | D 7 | D 28 |
| | Damaged Untreated Score % of Categories | | | | |
| 5-7 | 14 | 57 | 29 | 0 | 0 |
| 2-4 | 57 | 14 | 43 | 29 | 0 |
| 0-1 | 29 | 29 | 29 | 71 | 100 |
| >0 | 100 | 100 | 100 | 86 | 14 |
| 0 | 0 | 0 | 0 | 14 | 86 |
| | Damaged Treated Score % of Categories | | | | |
| 5-7 | 0 | 43 | 14 | 0 | 0 |
| 2-4 | 57 | 14 | 43 | 0 | 0 |
| 0-1 | 43 | 43 | 43 | 100 | 100 |
| >0 | 86 | 86 | 71 | 71 | 0 |
| 0 | 14 | 14 | 29 | 29 | 100 |

Redness (Visual Expert Grading Scores; VEGS) were detected from a value of 0 (no redness detected) to a maximum observed value of 7. Categorical values represent the percentages of total subjects on each day within that range of scores. For example, on D0, 29% of subjects had a score of between 0-1, 57% had a score between 2-4, and 14% had a score of between 5-7, totaling 100%. If the total is slightly more than 100%, this is due to rounding errors. The last two rows for untreated and Delta-5® treated, show the groupings as 0 (no redness detected), or >0 (redness detected). No statistics were applied to this type of data. D0* represents the measurement of redness immediately after application of SLS at baseline. D1, D3, D7, and D28 represent the days after application of SLS. Treatment with Delta-5® oil began just after D1, so D3 represents 2 days treatment with Delta-5®. No redness measurements were made on D2.

### Overall Findings:

Delta-5® oil, containing 24% of the bioactive, unique fatty acid SA, was extrapolated to have a stability of at least one year at room temperature in a neat formulation once opened, on basis of accelerated shelf-life testing and empirical findings. Delta-5® was also not a contact sensitizer nor skin irritant basis RIPT testing. Delta-5® oil thus has suitable stability and safety for cosmetic usage. In terms of efficacy, Delta-5® likely improved healing of SLS irritant-induced skin injury basis decreases in TEWL, impedance, and redness (vs. untreated skin) on days 3, 7 and 28 (D28 for TEWL and impedance). Delta-5® may thus benefit skin diseases such as dermatitis and eczema; and in normal skin, may improve skin elasticity and firmness.

TEWL and impedance showed consistent responses for evaluating skin barrier repair in response to SLS damage herein, and as reported by others. See Agner 1992 and Nicander 2003, supra. With both methodologies, Delta-5® accelerated healing following SLS damage over untreated skin at days 3, 7, and 28.

SLS damage from 2% SLS was maximal on days 1 and 2, basis increased TEWL and impedance. We did not test for reactive hyper-hydration (acute TEWL- and impedance increases) potentially occurring immediately after SLS-exposure. See Gloor M, Senger B, Langenauer M, Fluhr J W. On the course of the irritant reaction after irritation with sodium lauryl sulphate. Skin Res Technol. 2004; 10(3):144-48. Other groups have similarly reported greatest increases in TEWL and skin surface water 1-2 days after SLS, with repair commencing on days 3-7. See Katsarou A, Davoy E, Xenos K, Armenaka M, Theoharides T C. Effect of an antioxidant (quercetin) on sodium-lauryl-sulfate-induced skin irritation. Contact Derm. 2000; 42(2):85-9; Park S J, Kim H O, Kim G I, Jo H J, Lee J O, Lee C H. Comparison of skin responses for irritation produced by benzalkonium chloride and sodium lauryl sulfate. Korean J Dermatol. 2005; 43:1454-60. SLS-damage was expected to be fully repaired by 28 days, but we found skin was not fully healed in all subjects at 28 days, based on TEWL. Similar to Delta-5®, ceramides were also reported to promote healing from SLS after even 28 days, suggesting benefits of longer term usage following an acute injury or irritant insult. See Huang H-C, Chang T-M. Ceramide 1 and ceramide 3 act synergistically on skin hydration and the transepidermal water loss of sodium lauryl sulfate-irritated skin. Int J Dermatol. 2008; 47(8):812-19. Others have reported TEWL to be elevated after 1-2% SLS for 9-14 days, the last time point measured. See Gloor 2004 and Park 2005, supra.

The greater reduction in redness on days 3 and 7 with Delta-5® vs. untreated SLS-damaged sites indicates a redness-reducing benefit temporally coinciding with improvements in TEWL and impedance during the healing process. Reductions in redness following Delta-5® usage is one of the most common customer testimonials reported. There was high variability in redness scores at all time points across individuals, suggesting differential development- and resolution of redness. Redness may have been more uniform had forearms been cleaned with gentle soap prior to SLS-application. Similar to our results, in experiments with 1-2% SLS given to healthy volunteers for 24 h on the forearm, changes to TEWL and skin redness persisted 7-14 days. See Gloor 2004, Katsarou 2000 and Park 2005, supra. In the future, more sensitive methods for assessing redness could be employed, including erythema-index, utilizing a hand-held spectrometer. See Jemec G B, Johansen J D. Erythema-index of clinical patch test reactions. Skin Res Technol. 1995; 1(1):26-9.

### Proposed Mechanisms of Action for Improvement in Barrier Function:

The amount and type of skin surface fatty acids may affect barrier function as assessed with TEWL following skin irritation damage; and have a role in the etiology of skin barrier dysfunction in diseases such as atopic dermatitis. See Zhou M, Gan Y, Yang M, He C, Jia Y. Lipidomics analysis of facial skin surface lipids between forehead and cheek: association between lipidome, TEWL, and pH. J Cosmet Dermatol. 2020; 19(10):2752-58; Yen C-H, Dai Y-S, Yang Y-H, Wang L-C, Lee J-H, Chiang B-L. Linoleic acid metabolite levels and transepidermal water loss in children with atopic dermatitis. Ann Allergy Asthma Immunol. 2008; 100(1):66-73. We will explore below how topical application of SA could affect barrier function repair via proposed structural and signaling roles.

### Alteration of Lipids in the Extracellular Spaces of Stratum Corneum:

Permeability barrier of skin is mediated primarily by lipid-enriched lamellar membranes localized to extracellular spaces of stratum corneum. This unique structure contains 50% ceramides 1-6 (ceramides 1 and 3 being most important in barrier function disease etiology), 25% cholesterol, 15% free fatty acids, and very little phospholipid [13, 14, 36-40]. Bouwstra 2001 and Oh 2017, supra; Berthaud F, Boncheva M. Correlation between the properties of the lipid matrix and the degrees of integrity and cohesion in healthy human stratum comeum. Exp Dermatol. 2011; 20(3):255-62; Feingold K R, Elias P M. Role of lipids in the formation and maintenance of the cutaneous permeability barrier. Biochim Biophys Acta. 2014; 1841(3):280-94; van Smeden J, Janssens M, Gooris G S, Bouwstra J A. The important role of stratum corneum lipids for the cutaneous barrier function. Biochim Biophys Acta. 2014; 1841(3):295-313; Uchiyama M, Oguri M, Mojumdar E H, Gooris G S, Bouwstra J A. Free fatty acids chain length distribution affects the permeability of skin lipid model membranes. Biochim Biophys Acta. 2016; 1858(9):2050-59; Ourisman J, Barr K. Skin barrier: ingredients to support yours+why it's important. mbglifestyle. 2020. On the world wide web at mindbodygreen.com/articles/skin-barrier-ingredients-support-yours-why-its-so-important. Delta-5® oil could affect ceramides, free fatty acid levels, and phospholipid acyl composition in the lamellar membrane (See Berger 2002, supra); and this in turn could affect skin impedance and TEWL. See Bjorklund S, Ruzgas T, NowackaA, Dahi I, Topgaard D, Sparr E, et al. Skin membrane electrical impedance properties under the influence of a varying water gradient. Biophys J. 2013; 104(12):2639-50; Pappas A, Fantasia J, Chen T. Age and ethnic variations in sebaceous lipids. Dermatoendocrinol. 2013; 5(2):319-24. Of the fatty acids in Delta-5®, linoleic acid (42% of fatty acids detected in Delta-5®), palmitic acid (4%), stearic acid (2%) and 20:1n-9 (2%) could be incorporated into structural ceramides. Linoleic acid would only be incorporated into ceramides 1 and 4, linked to an omega-hydroxy fatty acid [43]. See Jungersted J M, Hellgren L I, Jemec G B, Agner T. Lipids and skin barrier function—a clinical perspective. Contact Derm. 2008; 58(5):255-62. As SA is not incorporated into sphingomyelin, the sphingosine base being common to sphingomyelins and ceramide, we would not expect SA to be a component of ceramides. See Berger A, Fenz R, German J B. Incorporation of dietary 5,11,14-icosatrienoate into various mouse phospholipid classes and tissues. J Nutr Biochem. 1993; 4:409-20.

### Effects on the Free Fatty Acid Pool and Stratum Corneum pH:

Following skin surface lipase action on the triacylglycerols present in Delta-5®, the free fatty acid pool generated could affect the acidic pH of approximately 4-5.5 at the stratum corneum surface and influence phase behavior. Jungersted 2008, supra.

Incorporation into keratinocyte phospholipid membranes: Fatty acids in keratinocyte phospholipid membranes are either synthesized endogenously or derived from extra-cutaneous sources. Feingold 2014, supra. Indeed, SA and other fatty acids in Delta-5® including linoleic acid (42%) and potentially its 2-carbon elongation product 20:2n-6 (11%) via retroconversion may be incorporated into keratinocyte membranes. Berger 2002, supra. Increases in lipid synthesis and lamellar body secretions are generally accepted to improve skin barrier function and permeability. See Kim M, Jung M, Hong S P, Jeon H, Kim M J, Cho M Y, et al. Topical calcineurin inhibitors compromise stratum comeum integrity, epidermal permeability and antimicrobial barrier function. Exp Dermatol. 2010; 19(6):501-10. SLS alters the repair phase of human skin via alteration of keratinocyte differentiation markers, and changes in enzymes degrading comeodesmosomes (intercellular adhesive structures in the stratum comeum). See Torma H, Lindberg M, Berne B. Skin barrier disruption by sodium lauryl sulfate-exposure alters the expressions of involucrin, transglutaminase 1, profilaggrin, and kallikreins during the repair phase in human skin in vivo. J Invest Dermatol. 2008; 128(5):1212-9. Following SLS-damage, fatty acid transport is increased by epidermal cytosolic fatty acid binding proteins, increasing fatty acids such as those present in Delta-5® in extracellular spaces. See Schurer NY Implementation of fatty acid carriers to skin irritation and the epidermal barrier. Contact Derm. 2002; 47(4):199-205.

### Anti-Inflammatory Signaling Roles of SA:

Upon release from cell membrane phospholipid pools, SA may also have important anti-inflammatory signaling roles. In cultured human skin keratinocytes, SA was not only incorporated into keratinocyte membranes, but also reduced levels of pro-inflammatory ARA and its pro-inflammatory down-stream mediator prostaglandin E2 (PGE2). See Berger 2002, supra. When SLS was applied to human skin in vivo, it induced differential expression of cyclooxygenase-2, the enzyme involved in synthesis of PGE2. See Clemmensen A, Andersen K E, Clemmensen O, Tan Q, Petersen T K, Kruse T A, et al. Genome-wide expression analysis of human in vivo irritated epidermis: differential profiles induced by sodium lauryl sulfate and nonanoic acid. J Investig Dermatol. 2010; 130(9):2201-10. Fatty acids such as SA can also affect gene transcription; and bind to receptors in cell signaling cascades. See Berger A, Roberts M A, Hoff B. How dietary arachidonic- and docosahexaenoic-acid rich oils differentially affect the murine hepatic transcriptome. Lipids Health Dis. 2006; 5(l):10; McCusker M M, Grant-Kels J M. Healing fats of the skin: the structural and immunologic roles of the omega-6 and omega-3 fatty acids. Clin Dermatol. 2010; 28(4):440-51; Khmaladze I, Leonardi M, Fabre S, Messaraa C, Mavon A. The skin interactome: a holistic “genome-microbiome-exposome” approach to understand and modulate skin health and aging. Clin Cosmet Investig Dermatol. 2020; 13:1021-40. In particular, the peroxisome proliferator-activated receptor PPAR is activated by some fatty acids including linoleic acid present in Delta-5® oil, leading to anti-inflammation in skin. See Schurer NY Implementation of fatty acid carriers to skin irritation and the epidermal barrier. Contact Derm. 2002; 47(4):199-205. In irritant contact dermatitis, PPAR agonists accelerate barrier recovery and enhance lamellar body synthesis, and neutral lipid synthesis (ceramides, cholesterol). Id. Our earlier work with keratinocytes demonstrated however that at least unmetabolized SA does not bind to PPAR receptors. See Berger 2002, supra. SA could also have cannabimimetic actions that affect skin inflammation and healing. Nakane S, Tanaka T, Satouchi K, Kobayashi Y, Waku K, Sugiura T. Occurrence of a novel cannabimimetic molecule 2-sciadonoylglycerol (2-eicosa-5′,11′,14′-trienoylglycerol) in the umbrella pine Sciadopitys verticillata seeds. Biol Pharm Bull. 2000; 23(6):758-61; Gachet M S, Schubert A, Calarco S, Boccard J, Gertsch J. Targeted metabolomics shows plasticity in the evolution of signaling lipids and uncovers old and new endocannabinoids in the plant kingdom. Sci Rep. 2017; 7:41177; Scheau C, Badarau I A, Mihai L-G, Scheau A-E, Costache D O, Constantin C, et al. Cannabinoids in the Pathophysiology of Skin Inflammation. Molecules (Basel, Switzerland). 2020; 25(3):652.

Delta-5® Oil May have Barrier Repair Emollient and Humectant Properties:

As a highly spreadable, cosmetic-grade, refined oil, Delta-5® could act as a barrier repair emollient and have humectant (moisturizing) properties, independent of the SA content. Emollients can decrease pro-inflammatory cytokines in skin with damaged epidermis, so it is feasible that the oil components of Delta-5® and SA both act to improve barrier function and barrier layer recovery from injury and irritation. See Ye L, Mauro T M, Dang E, Wang G, Hu L Z, Yu C, et al. Topical applications of an emollient reduce circulating pro-inflammatory cytokine levels in chronically aged humans: a pilot clinical study. J Eur Acad Dermatol Venereol. 2019; 33(11):2197-201. To test the specific biological activity of SA would require that another skin site be treated with purified SA, or with a control oil with all fatty acid components equivalent to those in Delta-5®, excepting the SA content. In rodent oral feeding studies, we demonstrated that it was the SA component of conifer oils rich in SA that displaced ARA from lipid pools, utilizing such an approach, exerting anti-inflammatory properties. See Berger 1993, supra; Berger A, German J B. Extensive incorporation of dietary D-5,11,14 eicosatrienoate into the phosphatidylinositol pool. Biochim Biophys Acta. 1991; 1085:371-76. Moreover, purified SA has been demonstrated in various models, including skin models, to be biologically active and anti-inflammatory on its own. See Berger 2002, supra.

Delta-5® Vs. Other Therapies to Improve Barrier Function:

An advantage of SA over steroidal and non-steroidal anti-inflammatory agents, and other drugs, for treating diseases with barrier layer dysfunction is that SA usage should not result in any tapering of benefits, and there should not be any adverse side effects (none observed amongst customers of Delta-5® oil and in pre-clinical studies to date). Topical glucocorticoids may lead to detrimental decreases in levels of stratum comeum lipids, countered by ultraviolet light; there is no reason to expect therapies with SA should have this negative effect. See Jungersted 2008, supra.

Most cosmetic products developed for improving condition of the barrier layer act as emollients and humectants. As previously noted, Delta-5®, may share these beneficial properties, with the additional benefit of SA acting in specific pathways to decrease inflammation associated with barrier layer dysfunction. Delta-5® would likely be even more effective if penetration enhancers or emulsifiers were added in an oil-in-water- or water-in-oil emulsion. An emulsion would additionally provide oxidative stability to the labile components in Delta-5 oil, such as SA.

Delta-5 oil (which consists of triacylglycerols) may not penetrate skin surfaces optimally, unless the skin surface is damaged, as in SLS-damaged skin. There must also be sufficient skin surface lipase activity from the host or microbes to catabolize the triacylglycerols in Delta-5® oil. We observed that Delta-5® penetrated the skin more efficiently following electroporation with a portable ultrasound device (Unpublished results). Another strategy to improve penetration would be to administer topical SA or oils rich in SA in the form of ethyl esters. Ethyl esters are approved for cosmetic use, commercially feasible to manufacture, and oxidatively stable. In previous work, we administered the related methyl esters of Delta-5® to mouse ears, and demonstrated penetration of SA and other fatty acids into the mouse ear skin; Delta-5® also reduced edema in the model system. See Berger 2002, supra.

### Systemic Benefits of Delta-5® Oil

Epidermal and skin barrier layer dysfunction can lead to development of chronic, low-grade systemic inflammation and inflammation associated with aging (so-called “inflammaging”) and associated systemic disorders, particularly in aging. See Haque A, Woolery-Lloyd H. Inflammaging in dermatology: a new frontier for research. J Drugs Dermatol. 2021; 20(2):144-49; Pilkington S M, Bulfone-Paus S, Griffiths C E M, Watson R E B. Inflammaging and the skin. J Invest Dermatol. 2021; 141(4s):1087-95. Correction of epidermal dysfunction with topical Delta-5 could thus be valuable for ameliorating aging-associated systemic disorders. As SA also has anti-inflammatory benefits when consumed orally based on rodent models, a combined strategy consisting of topical- and oral SA could be particularly beneficial for combatting inflammaging. It will also be valuable to determine if SA/Delta-5 oil effects the vast cutaneous microbiome, perturbations of which can affect inflammation. Ells 2012, supra.

### 4. Conclusion

Delta-5® is a novel, efficacious new oil to be used alone and with other ingredients to improve condition of the all-important skin barrier.

Knowing Delta-5® has healing benefits for SLS-induced skin irritation, paves way for other irritation and barrier function studies. Preventative benefits could be determined by applying Delta-5® prior to injury. See, Vie K, Cours-Dame S, Vienne M P, Boyer F, Fabre B, Dupuy P. Modulating effects of oatmeal extracts in the sodium lauryl sulfate skin irritancy model. See Skin Pharmacol Appl Skin Physiol. 2002; 15(2):120-4. Irritants could be evaluated alone or in combination with other substances including toxic plants, insect irritants, and the anti-acne ingredient benzoyl peroxide. See Weber S U, Thiele J J, Han N, Luu C, Valacchi G, Weber S, et al. Topical α-tocotrienol supplementation inhibits lipid peroxidation but fails to mitigate increased transepidermal water loss after benzoyl peroxide treatment of human skin. Free Radic Biol Med. 2003; 34(2):170-76. Additional conditions manifested by lipid barrier dysfunction could be probed including dermatitis, acne, eczema, and skin scarring. Delta-5® may also accelerate healing from burns, mechanical injury, environmental pollutants, allergens, and wounds (by accelerating wound closure and mitigating pain). By influencing barrier function, Delta-5® could also affect permeation of topical drugs and bioactive substances through skin outer layers. In normal skin, barrier function has roles in improving skin moisturization, structure, elasticity, firmness, frown lines and collagen production; and Delta-5® has a potential positive role to play as supported by relevant clinical testimonials.

### Abbreviations

ARA, arachidonic acid; D, Day; Delta-5, Delta-5 oil; DPMIU, Dermal Phase Meter impedance units; RBDW, refined, bleached, deodorized, and winterized; RIPT, Repeat Insult Patch Test; SA, sciadonic acid; SLS, sodium lauryl sulfate; TEWL, transepidermal water loss; VEG, Visual Expert Grading; VEGS, Visual Expert Grading Scores.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the technology as claimed should not be unduly limited to such specific embodiments. Indeed, various modifications of the described modes for carrying out the technology that are obvious to those skilled in pharmacology, biochemistry, medical science, or related fields are intended to be within the scope of the following claims.

We claim:

1. NMIFA composition for use in treating a disease or condition associated with loss of barrier function in a human or animal subject or to increase collagen production.

2. Use of claim 1, wherein the disease or condition associated with loss of barrier function in a human or animal subject is selected from the group consisting of inflammation or irritation caused by an allergen, inflammation or irritation caused by a chemical irritant, inflammation or irritation caused by nickel, latex, skin disinfectants, topical corticosteroids/glucocorticoids, topical germicidal compositions, inflammation or irritation caused by plant toxins or mechanical plant irritants, inflammation or irritation caused by insect toxins, inflammation or irritation caused by exposure to an oil-in-water (O/W) emulsion, inflammation or irritation due sunburn or damage caused by UV-B exposure, inflammation or irritation due to exposure to a skin whitening, bleaching, or lightening agent, inflammation or irritation caused by environmental pollutant, skin surface dysbiosis-induced irritation, dry skin due to loss of skin moisture, loss of skin elasticity, skin wrinkling, and frown lines.

3. Use of claim 2, wherein the allergen is pet dander.

4. Use of claim 2, wherein the chemical irritant is selected from the group consisting of bezalkonium chloride (BAC), sodium lauryl sulfate (SLS), benzoyl peroxide, a PABA-based chemical, oxybenzones, benzophenones, cinnamates, dibenzoylmethanes, chlorine, N,N-Diethyl-meta-toluamide (DEET), an adhesive, a chemical fragrance, a chemical found in household cleaners (e.g., dish detergents, laundry detergent, window cleaners, furniture polish, drain cleaners and toilet disinfectants), residual solvent (e.g., xylene, toluene, trimethylbenzene, octanoic acid, nonanoic acid, decanoic acid, and n-propanol and other alcohols) and a soap.

5. Use of claim 2, wherein the mechanical plant irritant is selected from the group consisting of a plant thorn, plant spine, plant nettle, plant glochid, plant trichome, and sharp-edged leaves.

6. Use of claim 2, wherein the plant toxin is selected from the group consisting of urushiol, calcium oxalate, protoanemonin, isothiocyanates, bromelain, diterpene esters, alkaloids, naphthoquinone and plant-derived acids.

7. Use of claim 2, wherein the insect toxin is selected from the group consisting of formic acid and mosquito saliva.

8. Use of claim 2, wherein the O/W emulsion is selected from the group consisting of Aqueous Cream BP 2001, Clioquinol Cream BP 1999 without clioquinol, Nonionic Hydrophilic Cream DAB 2001 without glycerol, Hydrophilic Skin Emulsion Base NRF, and Base Cream DAC.

9. Use of claim 2, wherein the skin whitening, bleaching, or lightening agent is selected from the group consisting of magnesium-1-ascorbyl-2-phosphate (MAP), hydroxyanisole, N-acetyl-4-S-cysteaminylphenol, arbutin (hydroquinone-beta-d-glucopyranoside) and hydroquinone.

10. Use of claim 2, wherein the environmental pollutant is selected from the group consisting of solar ultraviolet radiation (UVR), a polycyclic aromatic hydrocarbon (PAH), a volatile organic compounds (VOC), nitrogen oxides (NOx), particulate matter (PM), and cigarette smoke.

11. Use of claim 2, wherein the skin surface dysbiosis-induced irritation is caused by a skin pathogen or disruption of the skin microbiome.

12. Use of any of claims 1 to 11, wherein the NMIFA composition comprises at least 2% w/w, at least 5% w/w, at least 10% w/w, or at least 20% w/w of NMIFAs.

13. Use of any of claims 1 to 11, wherein the NMIFA composition comprises from 1% to 40% w/w, 1% to 30% w/w, 5% to 40% w/w, 5% to 30% w/w, or 10% to 25% w/w of NMIFAs.

14. Use of any one of claims 1 to 13, wherein the NMIFAs in the NMIFA composition are selected from the group consisting of sciadonic acid, juniperonic acid, pinoleic acid, dihomopinoleic acid, coniferonic acid (5, 9, 12, 15 18:4) and synthetic fatty acids selected from the group consisting of 1, 11, 14, 17 20:4; 2, 11, 14, 17 20:4; 3, 11, 14 17 20:4; 4, 11, 14 17 20:4; 6, 11, 14 17 20:4; 7, 11, 14 17 20:4; 1, 9, 12, 15 18:4; 2, 9, 12, 15 18:4; 3, 9, 12, 15 18:4; 4, 9, 12, 15 18:4, 5, 9, 12, 15 18:4, 1, 11, 14 20:3; 2, 11, 14 20:3; 3, 11, 14 20:3; 4, 11, 14 20:3; 6, 11, 14 20:3; 1, 9, 12 18:3; 2, 9, 12 18:3; 3, 9, 12 18:3; and 4, 9, 12 18:3 fatty acids and combinations thereof.

15. Use of any one of claims 1 to 14, wherein the NMIFA is sciadonic acid.

16. Use of any one of claims 1 to 15, wherein the NMIFAs in the NMIFA composition are provided as free fatty acids, esters, ethyl esters, triglycerides or phospholipids and combinations thereof.

17. Use of any one of claims 1 to 16, wherein the NMIFAs in the NMIFA composition are formulated with an antioxidant not naturally occurring with the NMIFAs.

18. Use of any one of claims 1 to 17, wherein the NMIFA composition is formulated for topical delivery.

19. Use of any one of claims 1 to 17, wherein the NMIFA composition is formulated for oral delivery vehicle.
